SEQUENCE LISTING (7 sequences)

SEQ ID NO: 1 - protein, 20 aa, Homo sapiens
VPDSCCINVTVGCGINFNEK

SEQ ID NO: 2 - protein, 9 aa, Homo sapiens
DVLETFTVK

SEQ ID NO: 3 - protein, 20 aa, Homo sapiens
QFYDQALQQAVVDDDANNAK

SEQ ID NO: 4 - protein, 15 aa, Homo sapiens
DVEMGNSVIEENEMK

SEQ ID NO: 5 - protein, 16 aa, Homo sapiens
LDCQVQGRPQPEVTWR

SEQ ID NO: 6 - protein, 16 aa, Homo sapiens
FGDPLGYEDVIPEADR

SEQ ID NO: 7 - protein, 12 aa, Homo sapiens
ALVGDEVELPCR

(19) United States
(12) Patent Application Publication
(10) Pub. No.: US 2025/0333692 A1
(43) Pub. Date: Oct. 30, 2025

(54) NANOCOMPOSITE INCLUDING MAGNETIC NANOPARTICLE COATED WITH PEPTIDE-IMPRINTED POLYMER, METHOD OF PREPARING THE SAME, AND USE THEREOF

(71) Applicant: UIF (University Industry Foundation), Yonsei University, Seoul (KR)

(72) Inventors: Yong SHIN, Seoul (KR); Hyo Joo LEE, Seoul (KR); Yeonjeong ROH, Busan (KR)

(21) Appl. No.: 19/187,023

(22) Filed: Apr. 23, 2025

(30) Foreign Application Priority Data
Apr. 29, 2024 (KR) 10-2024-0056672
Apr. 1, 2025 (KR) 10-2025-0042190

(51) Int. Cl. C12N 5/0793; C12N 5/079; C12N 13/00

(52) U.S. Cl. CPC C12N 5/0619; C12N 5/0622; C12N 13/00; C12N 2509/00

(57) ABSTRACT

Embodiments of the present disclosure herein relate to a nanostructure for isolating or concentrating extracellular vesicles (EVs), the nanostructure including a magnetic nanoparticle and a peptide-imprinted polymer; a method of isolating EVs using the nanostructure; and a method of preparing the nanostructure. The nanostructure according to the present invention includes a binding site of an EV protein, so it has selectivity for EVs, and when it includes a polymer imprinted with a peptide having an amino acid sequence of a specific cell-derived EV protein, it specifically isolates specific cell-derived EVs, and thus it is possible to selectively concentrate or isolate EVs with excellent efficiency in a short time. In addition, since a peptide, which is smaller in size and easier to synthesize than proteins, is used as a template, the nanostructure can be prepared simply at low cost.

### CROSS-REFERENCE TO RELATED APPLICATION

This application claims priority to and the benefit of Korean Patent Application No. 10-2024-0056672, filed on Apr. 29, 2024, the disclosure of which is incorporated herein by reference in its entirety.

This application claims priority to and the benefit of Korean Patent Application No. 10-2025-0042190, filed on Apr. 1, 2025, the disclosure of which is incorporated herein by reference in its entirety.

### SEQUENCE LISTING

This application contains references to amino acid sequences and/or nucleic acid sequences which have been submitted concurrently herewith as the sequence listing XML file entitled “000061us_SequenceListing.XML”, file size 7,214 bytes, created on Apr. 23, 2025. The aforementioned sequence listing is hereby incorporated by reference in its entirety pursuant to 37 C.F.R. § 1.52(e)(5).

### FIELD

Embodiments of the present disclosure herein relate to a nanostructure for isolating or concentrating extracellular vesicles, the nanostructure including magnetic nanoparticles coated with a peptide-imprinted polymer, a method of preparing the same, and a method of isolating or concentrating extracellular vesicles using the same.

The present disclosure is supported by a grant from the Korea government (Ministry of Health and Welfare, MOHW), titled “Development of early diagnostic and progression monitoring technology for multiple sclerosis based on glial cell-specific signals using EPIN technology”, under the R&D Program for Rare Disease Diagnosis and Treatment Technology Development (Project No.: RS-2025-02192998) (Project Research Period: Apr. 1, 2025 to Dec. 31, 2028).

### BACKGROUND

Neurological disorders, which include neuroinflammatory and neurodegenerative diseases, progressively worsen over time, and thus reliable biomarkers are required for early diagnosis and monitoring disease progression. However, existing brain tissue biopsies, imaging tests, and cerebrospinal fluid tests have difficulties in collecting biological samples, require expensive equipment, and have limitations in identifying the fundamental cause of the diseases.

Diagnostic methods based on blood biomarkers have advantages such that the methods are economically feasible compared to conventional technologies and biological samples may be easily obtained. Proteins and miRNAs are used as biomarkers for neurological diseases. However, it has been reported that they are not able to specifically distinguish various neurological diseases and are susceptible to interference from other blood components such as degrading enzymes.

Neuromyelitis optica spectrum disorder (NMOSD) is an inflammatory disease of the central nervous system. NMOSD is an autoimmune disease that attacks the aquaporin-4 (AQP4) protein of astrocytes with autoantibodies. NMOSD is a lifelong disease with unpredictable symptoms such as optic neuritis and myelitis, and is accompanied by various neurological disorders and treatment responses. There are existing biomarkers for diagnosing NMOSD, such as AQP4 antibodies and glial fibrillary acidic protein, but they have problems such as inconsistent correlation with the disease and low accuracy.

Diagnosis methods for neurological diseases using brain-derived extracellular vesicles (EVs) are being actively studied. EVs secreted from cells are nano-sized vesicles secreted by cells into the extracellular environment to transmit information between cells in the body. EVs contain a large amount of biomarkers (genes, nucleic acids, proteins, etc.) of the cells from which they are derived, reflecting the state of the cells. In addition, EVs can pass through the blood-brain barrier, are found in various body fluids, and protect proteins and nucleic acids therein from degradation through the membrane of the vesicles. Therefore, diseases may be diagnosed and monitored in various ways through analysis of substances contained in the EVs as well as analysis of the EVs themselves. However, due to the complex nature of body fluids such as blood and the low concentration of EVs in samples, research is still needed to develop a technology for efficiently collecting specific EVs.

A method to overcome these limitations is to utilize nanoparticles that have strong binding affinity to EV (exosome) membrane proteins. It has been reported that exosomes may be captured using nanoparticles including aptamers or peptides that have strong binding affinity to exosome membrane proteins. In addition, molecular imprinting polymer (MIP) technology is a technology that forms a binding site for a template material by forming a polymer with a monomer and a crosslinker surrounding the template material, and it is simple, fast, and economical. In the MIP technology, a wide range of materials may be selected as templates, from small molecules to cells.

The method of imprinting an exosome itself may enable the preparation of nanoparticles having exosome binding sites, but as mentioned above, there is a problem due to the limitations of the technology for purifying exosomes. Therefore, proteins of the exosome can be selected as alternative template materials. However, proteins other than tetraspanin proteins of the exosome (such as CD63, CD9, and CD81) have structural complexity and difficulty in increasing purity.

### SUMMARY

Provided is a nanostructure that can be used for simple and easy isolation of extracellular vesicles (EVs).

Also provided is a method of isolating EVs using the nanostructure.

Also provided is a method of preparing the nanostructure.

The present inventors have discovered that by using a polymer on which a monomer is imprinted using a peptide having an amino acid sequence of an EV-derived membrane protein as a template and a magnetic nanoparticle, EVs may be isolated by binding to the EVs and by a magnetic force from a magnetic field, thereby completing the present invention.

An embodiment of the inventive concept includes a nanostructure for isolating or concentrating EVs, the nanostructure including a magnetic nanoparticle coated with a peptide-imprinted polymer.

In an embodiment, the magnetic nanoparticle and the peptide-imprinted polymer may be linked by an amide bond.

In an embodiment, the magnetic nanoparticle may be an iron oxide (Fe₃O₄) nanoparticle.

In an embodiment, the nanostructure may have a diameter of 100 to 200 nm.

In an embodiment, the peptide may consist of 8 to 20 amino acids.

In an embodiment, the peptide may be derived from an EV membrane protein of a brain cell.

In an embodiment, the brain cell may be at least one selected from an astrocyte, a neuron, a microglial cell, and an oligodendrocyte.

In an embodiment, the peptide may consist of any one of SEQ ID NOs: 4 to 7.

In an embodiment, the polymer may be polymerized from at least one monomer selected from styrene, N-(3-aminopropyl)methacrylamide, N-isopropylacrylamide, methacrylic acid, ethylene glycol dimethacrylate, N-tert-butylacrylamide, N,N-dimethylaminopropyl acrylamide, and acrylamide.

In an embodiment, at least one selected from acrylic acid, N,N-methylenebis(acrylamide), ammonium persulfate, N,N,N′,N′-tetramethylethylenediamine, and benzoyl peroxide may be further added during polymerization.

In an embodiment, the nanostructure may be for isolating or concentrating EVs in a blood or cell culture medium sample.

In an embodiment, the nanostructure may be for isolating or concentrating EVs derived from a brain cell.

An embodiment of the inventive concept includes a method of isolating or concentrating EVs using the nanostructure.

In an embodiment, a manufacturing method including: a step of bringing the nanostructure into contact with EVs; and a step of applying a magnetic field, may be provided.

In an embodiment, a manufacturing method further including: a step of isolating a material captured by the magnetic force from the magnetic field; and a step of isolating the EVs from the nanostructure, may be provided.

An embodiment of the inventive concept includes a method of preparing the nanostructure, the method including: (i) a step of treating a magnetic nanoparticle with ammonia water; (ii) a step of treating the product of Step (i) with a peptide; (iii) a step of treating the product of Step (ii) with a monomer compound; (iv) a step of polymerizing the monomer compound of the product of Step (iii) into a polymer; and (v) a step of removing the peptide from the product of Step (iv).

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a schematic diagram illustrating a method of preparing a nanostructure (EPIN) of the present invention and a drawing illustrating a method of isolating or concentrating extracellular vesicles (EVs);

FIG. 2 shows scanning electron microscope (SEM) images comparing the uniform particle size distribution and shape of the nanostructure (EPIN) of the present invention with protein-imprinted molecular imprinting polymer (MIP) nanoparticles;

FIG. 3 shows the results of comparing the particle size of the nanostructure (EPIN) of the present invention with protein-imprinted MIP nanoparticles.

FIG. 4 shows the results of comparing the zeta potential of the nanostructure (EPIN) of the present invention with protein-imprinted MIP nanoparticles;

FIG. 5 shows the results of the Fourier transform infrared spectroscopy (FT-IR) spectra measured at each stage of synthesizing the nanostructure (EPIN) of the present invention;

FIGS. 6 and 7 show the results of comparing the EV isolation performance (EV capture efficiency and miRNA expression level in EVs) of the nanostructure (EPIN) of the present invention with protein-imprinted MIP nanoparticles;

FIG. 8 shows the results of comparing the EV isolation performance (miRNA expression level in EVs) of the nanostructure (EPIN) of the present invention with ultracentrifugation (UC), a commercially available exosome isolation kit (Exo-isolation kit (EI), and protein-imprinted MIP nanoparticles;

FIG. 9 shows the results of comparing the EV isolation performance (miRNA expression level in EVs) of the nanostructure (EPIN) of the present invention according to the sample volume;

FIG. 10 shows SEM images illustrating the distribution and shape of EVs isolated by the nanostructure (EPIN) of the present invention;

FIG. 11 shows a transmission electron microscope (TEM) image illustrating the distribution and shape of EVs isolated by the nanostructure (EPIN) of the present invention;

FIG. 12 shows the results of comparing the particle size of EVs isolated by the nanostructure (EPIN) of the present invention with EVs isolated by protein-imprinted MIP nanoparticles;

FIG. 13 shows the results of comparing the zeta potential of EVs isolated by the nanostructure (EPIN) of the present invention with EVs isolated by protein-imprinted MIP nanoparticles;

FIG. 14 is a graph illustrating the distribution of the tetraspanin family, which is a membrane protein, in EVs isolated by the nanostructure (EPIN) of the present invention;

FIG. 15 shows the results of comparing EVs isolated by the nanostructure (EPIN) of the present invention with EVs isolated by protein-imprinted MIP nanoparticles by Western blotting;

FIG. 16 shows the results of comparing the zeta potential of the nanostructure (EPIN-GLAST) of the present invention with non-imprinted magnetic nanoparticles (MNP);

FIG. 17 shows the results of analyzing EVs isolated by the nanostructure (EPIN-GLAST) of the present invention from cell culture media of astrocyte-like cell lines and colorectal cancer cell lines by Western blotting;

FIG. 18 shows the results of analyzing EV-derived proteins isolated from blood samples of neuromyelitis optica spectrum disorder (NMOSD) patients and EV-derived proteins isolated from blood samples of non-patients using the nanostructure (EPIN-GLAST) of the present invention by Western blotting;

FIG. 19 shows the results of analyzing the expression levels of miRNA extracted from EVs isolated from blood samples of NMOSD patients and miRNA extracted from EVs isolated from blood samples of non-patients using the nanostructure (EPIN-GLAST) of the present invention by RT-qPCR;

FIG. 20 shows the results of confirming the changes in the graphs of EPINs prepared with various templates using FT-IR; and

FIG. 21 shows the results of confirming EVs cell-specifically isolated using EPIN-MOG, EPIN-L1CAM, and EPIN-CD11b using Western blotting.

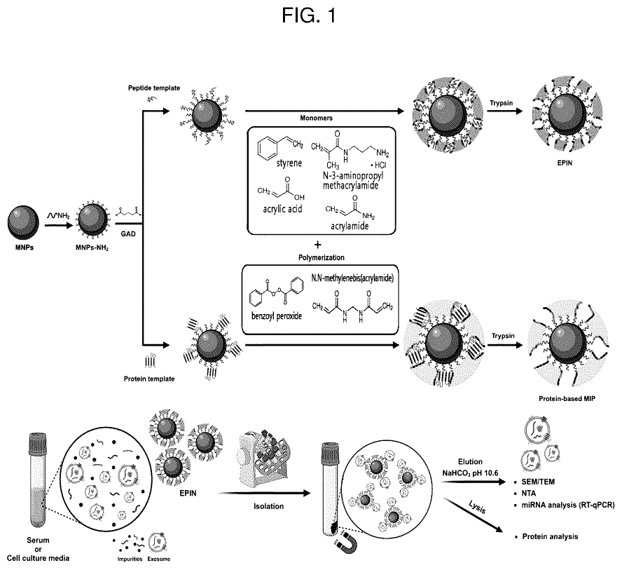

### DETAILED DESCRIPTION

Hereinafter, the present invention will be described in detail.

An embodiment of the inventive concept includes a nanostructure (engineered polymer-inorganic nanocomposites, EPIN) for isolating or concentrating extracellular vesicles (EVs), the nanostructure including a magnetic nanoparticle coated with a peptide-imprinted polymer.

The term “magnetic nanoparticle” (MNP) is not particularly limited as long as it is a nano-sized particle that is capable of responding to a magnetic field and binding to an organic substance through surface modifications.

In an embodiment, the magnetic nanoparticle may be an iron oxide (Fe₃O₄) nanoparticle.

In an embodiment, the nanostructure may have a diameter of 100 to 200 nm.

The term “peptide-imprinted polymer” refers to a polymer in which a monomer surrounds a peptide to form a polymer and then an imprinted binding site is formed when a template material is removed.

The term “monomer” as used herein refers to a material that forms a covalent bond or a non-covalent bond with a peptide to synthesize a peptide-imprinted polymer and forms a polymer through polymerization.

The term “Extracellular vesicle (EV)” includes all nano-sized (30 to 2,000 nm) vesicles released outside the cell, which are composed of a phospholipid bilayer, which is the same component as the structure of the cell membrane. Therefore, EVs include “exosomes” and “microvesicles” released from cells. Furthermore, the term “EV” is also used to refer to ectosomes, microparticles, tolerosomes, prostatosomes, cardiosomes, and vexosomes.

The term “nanostructure” refers to a very small particle having a nanometer size and capable of binding to EVs, as it is formed as a complex of a magnetic nanoparticle and a peptide-imprinted polymer.

In an embodiment, the magnetic nanoparticle and the peptide-imprinted polymer may be linked by an amide bond, and the peptide may consist of 8 to 20 amino acids.

In an embodiment, the brain cell may be at least one selected from an astrocyte, a neuron, a microglial cell, and an oligodendrocyte.

In an embodiment, the peptide may consist of any one of SEQ ID NOs: 4 to 7 as shown in Table 1 below. Hereinafter, a nanostructure using the glutamate aspartate transporter 1 (GLAST) peptide sequence is abbreviated as EPIN-GLAST. CD63, CD9, and CD81 below are tetraspanin proteins that are generally present in the EV membrane, and were used to optimize EPIN preparation according to one embodiment of the present invention and to confirm the EV isolation efficiency from the cell culture medium.

TABLE 1

| | | | Peptide | |
|---|---|---|---|---|
| | | | length | |
| Target | Protein | Peptide sequence | [aa] | SEQ ID NO: |
| EVs derived from all cells | CD63 | VPDSCCINVTVGCGINFNEK | 20 | SEQ ID NO: 1 |
| | CD9 | DVLETFTVK | 9 | SEQ ID NO: 2 |
| | CD81 | QFYDQALQQAVVDDDANNAK | 20 | SEQ ID NO: 3 |
| EVs derived from astrocytes | GLAST(EAAT1) | DVEMGNSVIEENEMK | 15 | SEQ ID NO: 4 |
| EVs derived from neurons | L1CAM | LDCQVQGRPQPEVTWR | 16 | SEQ ID NO: 5 |
| EVs derived from microglia | CD11b(ITAM) | FGDPLGYEDVIPEADR | 16 | SEQ ID NO: 6 |
| EVs derived from oligodendrocytes | MOG | ALVGDEVELPCR | 12 | SEQ ID NO: 7 |

In Table 1 above, GLAST (excitatory amino acid transporter 1 (EAAT1)) is a protein that is located in an astrocyte-derived EV cell membrane and removes glutamate from the extracellular space; L1 cell adhesion molecule (L1CAM) is a neuronal cell adhesion protein that is located in a neuron-derived EV cell membrane and strongly affects cell migration, adhesion, neurite outgrowth, myelination, and neural differentiation; CD11b (integrin αM (ITAM)) is a protein that is located in a microglial cell-derived EV cell membrane and regulates the migration of neutrophils to sites of infection and inflammation; and myelin oligodendrocyte glycoprotein (MOG) is a protein that is located in an oligodendrocyte-derived EV cell membrane and regulates the myelination of central nervous system neurons.

The peptide used in the present invention may include not only a peptide but also a derivative thereof. For example, the peptide of the present invention may include a peptide having 80% or more homology, preferably 90% or more homology, more preferably 95% or more homology with the peptide of each corresponding sequence number, and the derivative may include a peptide in which the N-terminus, C-terminus, and the like of the peptide are chemically modified or an amino acid is added, substituted, or deleted, and is not particularly limited.

In an embodiment, the polymer may be polymerized from at least one monomer selected from styrene, N-(3-aminopropyl)methacrylamide, N-isopropylacrylamide, methacrylic acid, ethylene glycol dimethacrylate, N-tert-butylacrylamide, N,N-dimethylaminopropyl acrylamide, and acrylamide, but is not limited thereto. Any monomer capable of forming a polymer that can coat a magnetic nanoparticle by combining with a peptide is also included in the scope of the present invention.

In an embodiment, at least one selected from acrylic acid, N,N-methylenebis(acrylamide), ammonium persulfate, N,N,N′,N′-tetramethylethylenediamine, and benzoyl peroxide may be further added during polymerization, but it is not limited thereto. Any substance that is recognized to be capable of serving as a cross-linking agent when the monomer forms a polymer is also included in the scope of the present invention.

In an embodiment, the nanostructure may be for isolating or concentrating EVs in a blood or cell culture medium sample or may be for isolating or concentrating EVs derived from a brain cell.

An embodiment of the inventive concept includes a method of isolating or concentrating EVs using the nanostructure.

In an embodiment, a manufacturing method including: a step of bringing the nanostructure into contact with EVs; and a step of applying a magnetic field, may be provided.

The applying of the magnetic field may be performed, for example, by bringing a magnet into contact with a container containing target EVs.

In an embodiment, a method further including: a step of isolating a material captured by the magnetic force from the magnetic field; and a step of isolating the EVs from the nanostructure, may be provided.

In addition, the method of isolating the captured material may be performed by, for example, discharging remaining materials from the container except for a target material remaining in the container due to the magnet. The method of isolating the EVs from the nanostructure may be performed by, for example, performing a process of cutting linking portions on the nanostructure.

In addition, an embodiment of the inventive concept includes a method of preparing the nanostructure, the method including: (i) a step of treating a magnetic nanoparticle with ammonia water; (ii) a step of treating the product of Step (i) with a peptide; (iii) a step of treating the product of Step (ii) with a monomer compound; (iv) a step of polymerizing the monomer compound of the product of Step (iii) into a polymer; and (v) a step of removing the peptide from the product of Step (iv).

The terms used in the preparation method are consistent with the terms described for the above-described nanostructure.

Hereinafter the present invention will be described in more detail through examples. However, these examples are intended to exemplify the present invention, and the scope of the present invention is not limited to these examples.

### [Example 1] Synthesis of Nanostructure (EPIN) According to the Present Invention

1-1. Synthesis of Magnetic Nanoparticle (MNP-NH₂) Coated with Amino Group

About 600 mg of MNPs was mixed with 5 ml of 3-aminopropyltriethoxysilane (APTES), 5 mL of ammonia water (NH₄OH), and 200 ml of ethanol (99.99%), and the resulting mixture was sonicated for 30 minutes. Thereafter, the mixture was stirred at 80° C. for four hours, at 120° C. for two hours, and at 56° C. overnight. After washing several times with distilled water, the mixture was dried at 56° C. for two hours.

### 1-2. Peptide Design for EPIN Preparation

The amino acid sequence of a target protein was retrieved from http://www.uniprot.org/, and the sequence was used in the FASTA standard format. The FASTA sequence of a protein was cleaved (http://web.expasy.org/peptide_cutter/) using a desired cutting method (trypsin was used in this example). Among the cleaved peptides, peptides with 8 to 20 amino acids were selected. In addition, the intracellular and extracellular regions of the protein were confirmed through the structural diagram of the target protein. The selected peptides were sequenced using protein Basic Local Alignment Search Tool (BLAST) (blast.ncbi.nlm.nih.gov/Blast.cgi). As a result of the analysis, peptides with high total scores and low E-values were selected to select the most stable and sequence-specific peptides (see Table 2 below).

TABLE 2

| | | | Peptide | |
|---|---|---|---|---|
| | | | length | |
| Target | Protein | Peptide sequence | [aa] | SEQ ID NO: |
| EVs derived from all cells | CD63 | VPDSCCINVTVGCGINENEK | 20 | SEQ ID NO: 1 |
| | CD9 | DVLETFTVK | 9 | SEQ ID NO: 2 |
| | CD81 | QFYDQALQQAVVDDDANNAK | 20 | SEQ ID NO: 3 |
| EVs derived from astrocytes | GLAST(EAAT1) | DVEMGNSVIEENEMK | 15 | SEQ ID NO: 4 |
| EVs derived from neurons | L1CAM | LDCQVQGRPQPEVTWR | 16 | SEQ ID NO: 5 |
| EVs derived from microglia | CD11b(ITAM) | FGDPLGYEDVIPEADR | 16 | SEQ ID NO: 6 |
| EVs derived from oligodendrocytes | MOG | ALVGDEVELPCR | 12 | SEQ ID NO: 7 |

### 1-3. EPIN Synthesis

A nanostructure (EPIN) was synthesized using the monomer and the above-described MNP-NH₂ and peptide.

Briefly, a mixture of MNP-NH₂, the peptide, and 0.5% glutaraldehyde (GAD) was stirred overnight at 60 rpm. After removing the solution, the resulting mixture was washed several times with N-2-hydroxyethylpiperazine-N-2-ethane sulfonic acid (HEPES). A monomer (10³ ng/100 μl) such as styrene, N-(3-aminopropyl) methacrylamide (3APM), N-isopropylacrylamide (NIPPAM), methacrylic acid (MAA), ethylene glycol dimethacrylate (EGDMA), N-tert-butylacrylamide, N, N-dimethylaminopropyl acrylamide, or acrylamide was added, and the resulting mixture was stirred at 60 rpm for three hours. At this time, the peptide forms a covalent bond or a non-covalent bond with the monomer. Acrylic acid (103 ng/100 μl), N,N-methylenebis(acrylamide) (NMAA) (3×10² ng/300 μl), ammonium persulfate, N,N,N′,N′-tetramethylethylenediamine, or benzoyl peroxide (BPO) (0.5×10² ng/50 μl) was further added, and the resulting mixture was stirred at 60 rpm overnight for polymerization of the monomers. Thereafter, the solution was removed, and the product was incubated with trypsin at 37° C. and 1000 rpm for three hours. The resulting structures were washed with HEPES and then used.

Hereinafter, EPINs imprinted with peptides derived from CD63, CD9, CD81, GLAST, L1CAM, CD11b, and MOG are abbreviated as EPIN-CD63, EPIN-CD9, EPIN-CD81, EPIN-GLAST, EPIN-L1CAM, EPIN-CD11b, and EPIN-MOG, respectively.

### [Example 2] Confirmation of Structure Preparation and Evaluation of Characteristics

In order to confirm the characteristics of the peptide-imprinted nanostructure (EPIN) prepared in Example 1, the following experiments were performed.

### 2-1. Scanning Electron Microscope (SEM) Images

The distribution and shape of the nanostructure (EPIN) of the present invention were confirmed through SEM experiments. The nanostructure (EPIN) sample of the present invention was dropped on a silicon substrate, dried, and then coated with Pt before imaging in a SEM chamber (JSM-7800F). The size, shape, and distribution of 200 randomly selected particles were measured.

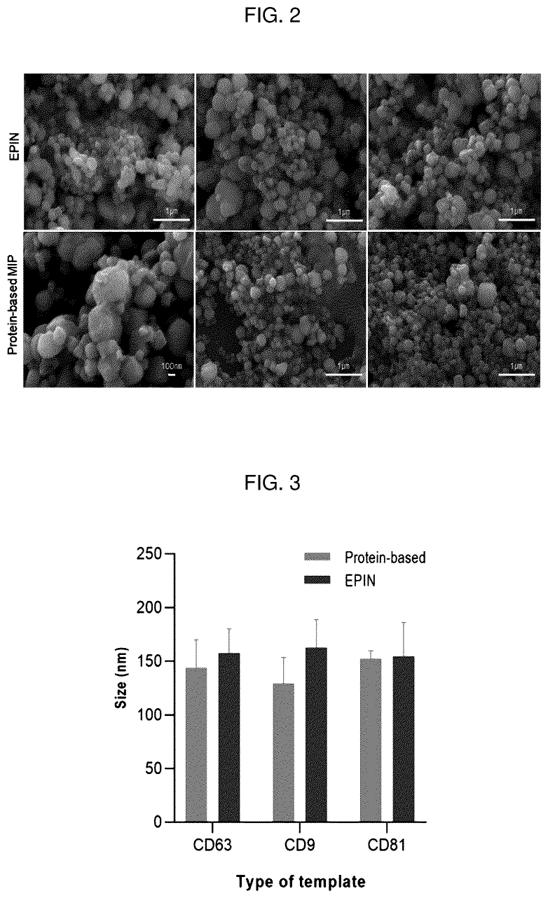

As a result, FIG. 2 shows SEM images comparing the uniform particle size distribution and shape of the nanostructure (EPIN) of the present invention with protein-imprinted MIP nanoparticles. From the SEM images, it can be confirmed that the nanostructure (EPIN) of the present invention has a similar shape to the existing protein-imprinted MIP nanoparticles and has a uniform particle size distribution and shape.

### 2-2. Size of Nanostructure

A nanoparticle tracking analysis was performed to confirm the size of the nanostructure (EPIN). The prepared nanostructure was visualized using a nanoparticle tracking analyzer (NanoSight NS300 device).

As a result, FIG. 3 shows images comparing the particle size of the nanostructure (EPIN) of the present invention with protein-imprinted MIP nanoparticles. It can be seen that EPIN is relatively larger than the protein-imprinted MIP nanoparticles. This may be because, although the peptide is smaller than the protein, the number of template peptides bound to the magnetic nanoparticles by amide bonds was greater, which affected polymer formation.

### 2-3. Zeta Potential

To measure the surface charge, that is, zeta potential, of EPIN, a Zetasizer Lab instrument (Malvern Instruments, UK) was employed. Zeta potential measurements were obtained by adding EPIN to 1 ml of distilled water at neutral pH of 7.0.

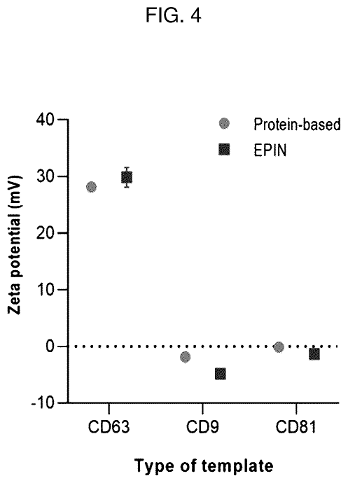

As a result, FIG. 4 shows the zeta potentials of the magnetic nanoparticles (MNP), the magnetic nanoparticles with amino groups exposed on the surface (MNP-NH₂), EPIN-CD63, EPIN-CD9, and EPIN-CD81. Different zeta potentials indicate that there was a change in the polymer formation process of the monomer bound to the peptide. In other words, it indicates that the polymer was imprinted according to each peptide type and coated on the magnetic particles.

### 2-4. Fourier Transform Infrared Spectroscopy (FT-IR) Spectrum

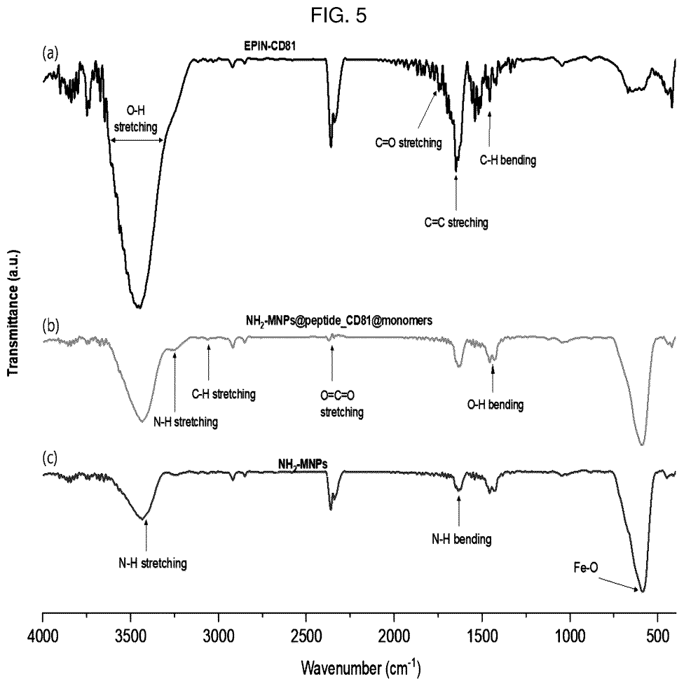

FIG. 5 shows a diagram illustrating the FT-IR spectra. Line (a) is EPIN-CD81, line (b) is MNP-NH₂ with the CD81 peptide and a monomer combined, and line (c) is MNP-NH₂. FIG. 5 indicates that EPIN-CD81 was synthesized, since there was a change in the peaks at each step.

### [Example 3] Conformation of EV Isolation

In order to perform EV isolation experiments using the peptide-imprinted nanostructures (EPIN) prepared in Example 1 and confirm characteristics such as isolation efficiency, the following experiments were performed.

### 3-1. Optimization of EPIN Preparation

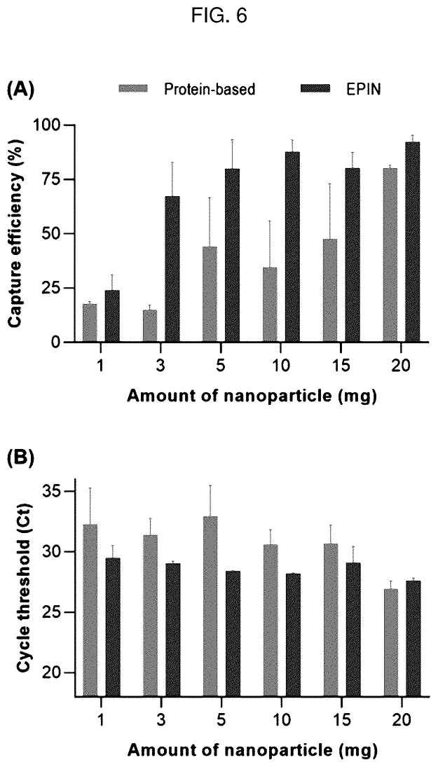

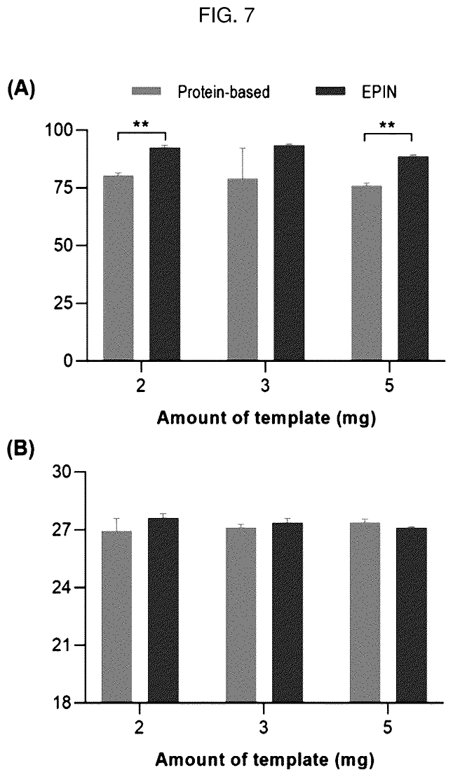

First, in order to confirm the EV isolation performance of the nanostructure (EPIN) of the present invention, it was compared with protein-imprinted MIP nanoparticles, and FIGS. 6 and 7 show the data on the capture efficiency (%) and miRNA expression level. EVs were isolated from the HCT116 (colorectal cancer cell line) cell culture medium, and the capture efficiency (FIGS. 6A and 7A) was confirmed using a nanoparticle tracking analyzer, and the miRNA (hsa-mir-21) expression level in the EVs was quantified using reverse transcription quantitative polymerase chain reaction (RT-qPCR) (FIGS. 6B and 7B).

As a result, FIG. 6A shows that EPIN exhibited sufficient EV isolation efficiency even with a smaller amount of magnetic nanoparticles than MIP, and FIG. 6B shows that the largest number of EVs were isolated when 20 mg of magnetic nanoparticles were used, as indicated by the low Ct value.

In addition, FIG. 7 shows that the isolation efficiency and Ct value according to the amount of template material have a negligible difference. Through this, the amount of magnetic nanoparticles and template peptide to optimize EPIN preparation may be determined.

Next, the miRNA (hsa-mir-21) expression level in EVs isolated using ultracentrifugation (UC), an exosome isolation kit (EI), protein-imprinted MIP nanoparticles, and EPIN was compared.

EVs were isolated from an HCT116 cell culture medium, and hsa-mir-21 was extracted from the EVs. The EV isolation efficiency by UC, EI, protein-imprinted MIP nanoparticles, and EPIN was compared by performing quantitative real-time PCR (qRT-PCR) for hsa-mir-21 and then using the Ct value.

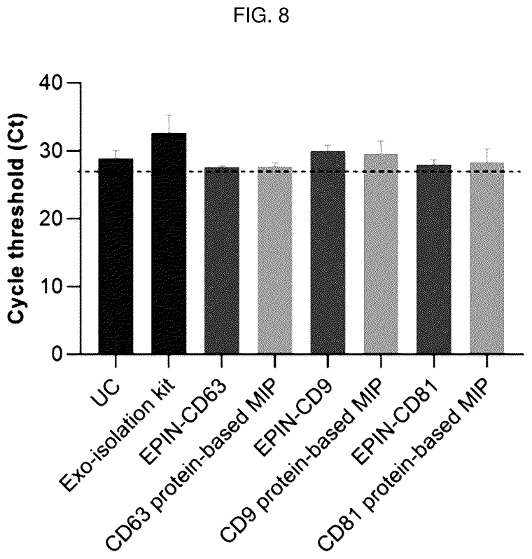

As a result, as shown in FIG. 8, hsa-mir-21 was recovered more efficiently from EVs isolated by EPIN than by EI. No significant difference was found in the Ct value when compared with UC or protein-imprinted MIP nanoparticles. These results suggest that EPIN may be used as a suitable method for EV isolation, comparable to UC or protein-imprinted MIP nanoparticle technologies.

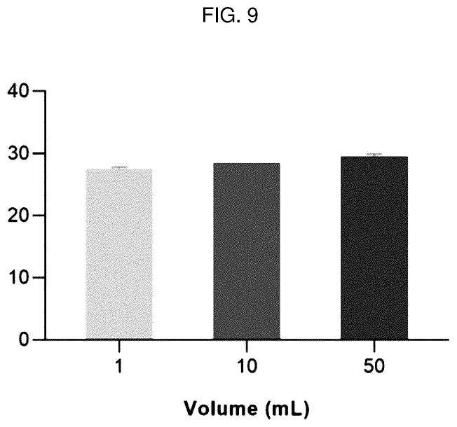

In addition, FIG. 9 shows the results of analyzing the EV isolation efficiency of EPIN according to the sample volume. The graph compares the Ct values obtained by performing qRT-PCR for hsa-mir-21 of EVs. From FIG. 9, it can be confirmed that EPIN was not affected by the sample volume.

The above-described series of results suggest that the EPIN according to the present invention is a simple and novel method capable of isolating EVs with higher efficiency and purity than the conventionally known technologies of UC, EI, and protein-imprinted MIP nanoparticles. In addition, since there is no need to use UC or a complex and slow device, it is possible to provide a simple and rapid method.

### 3-2. Analysis of EVs Isolated by EPIN

SEM and TEM were used to investigate the morphology of the isolated EVs and identify exosome markers. EVs were fixed in a paraformaldehyde solution, serially diluted in distilled water, and then photographed using a scanning electron microscope. Immunogold staining antibodies for target proteins were used to identify EV proteins and confirm the presence of EV membrane proteins that interact with EPIN.

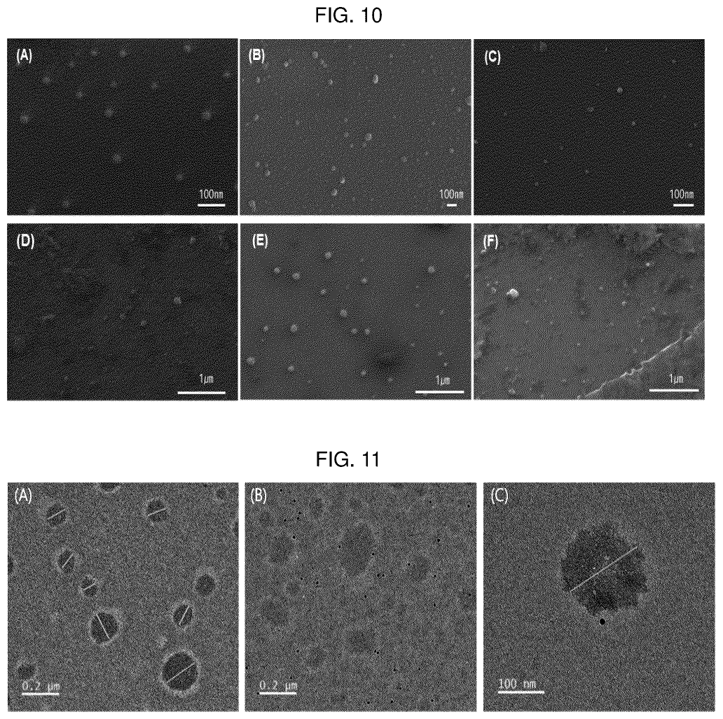

As a result, FIG. 10 shows SEM images of the circular vesicle morphology of the isolated EVs. FIGS. 10A to 10C show EVs isolated by protein-imprinted MIP nanoparticles, and FIGS. 10D to 10F show SEM images of EVs isolated by EPIN.

In addition, FIG. 11 shows the TEM images of the shape of EVs analyzed by labeling the isolated EVs with CD63-Immunogold. FIG. 11A shows a unlabeled control group, FIG. 11B shows EVs isolated by protein-imprinted MIP nanoparticles, and FIG. 11C shows EVs isolated by EPIN. From FIGS. 10 and 11, it can be seen that EVs isolated by EPIN are circular and have a size of less than 200 nm.

To measure the size distribution and concentration of the isolated EVs, a nanoparticle tracking analysis was performed. The eluted EVs were diluted with phosphate-buffered saline (PBS) and visualized on a nanoparticle tracking analyzer (NanoSight NS300 device).

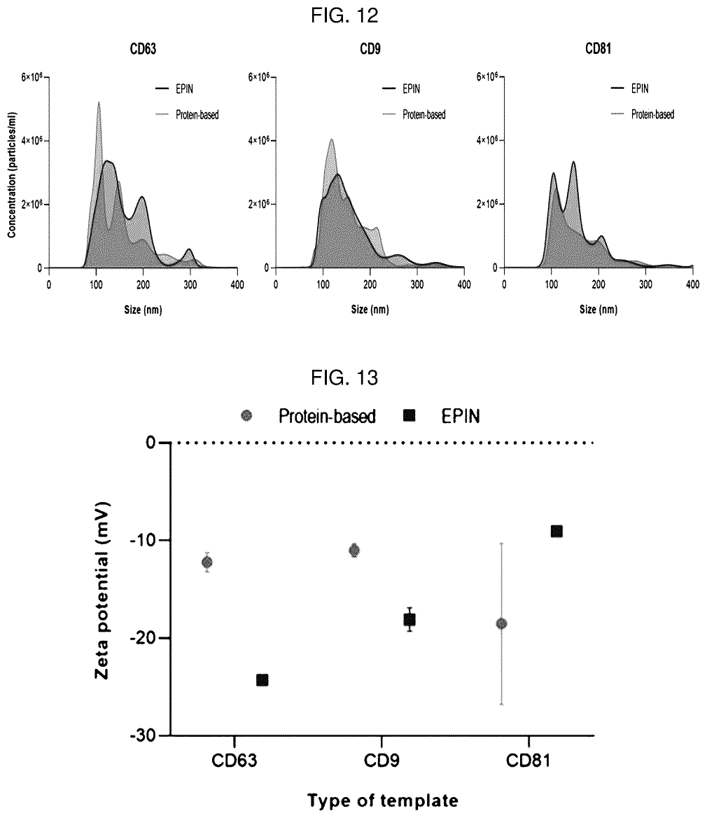

As a result, FIG. 12 shows data on the measured size and concentration of the isolated EVs. The distribution of EVs with a size of 200 nm or larger was rapidly reduced, confirming that even though the vesicle proteins binding to EPIN were different, what was isolated by each EPIN was EVs.

To confirm the surface charge of the isolated EVs, zeta potential was analyzed. Zeta potential was measured using Zetasizer Lab (Malvern Instruments, UK). Prior to measurement, EVs isolated using EPIN were mixed with 200 μL of elution buffer and diluted with distilled water to a total volume of 1 mL.

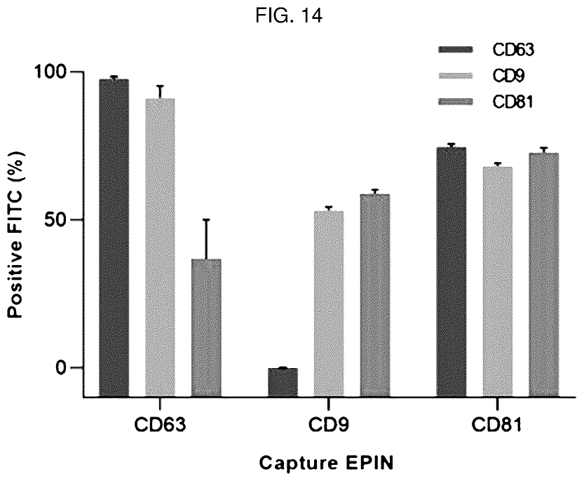

As a result, FIG. 13 shows the zeta potential of the isolated EVs. It can be confirmed that the isolated EVs have a negative potential similar to exosomes.

Flow cytometry was used to determine the distribution of CD63, CD9, and CD81 in the membrane of the isolated EVs. The isolated EVs were bound to each antibody (anti-CD63, CD9, and CD81 antibodies) (System Biosciences, CA, USA) conjugated to streptavidin beads according to the manufacture's protocol using the Tetraspanin Exo-Flow Combo Capture Kit (System Biosciences, CA, USA). The isolated EVs were then labeled with fluorescein isothiocyanate (FITC). Finally, samples were subjected to flow cytometry using a BD™ LSR II flow cytometer (BD Biosciences, Franklin Lakes, NJ, USA) and the data was analyzed using FlowJo software. Synthetic beads without EVs were used as a negative control.

As a result, FIG. 14 shows that the distribution of CD63, CD9, and CD81, which are membrane proteins of the isolated EVs, determined using flow cytometry differs depending on the target of EPIN.

Western blotting was performed to confirm the presence of target proteins distributed in the vesicle membrane of the isolated EVs.

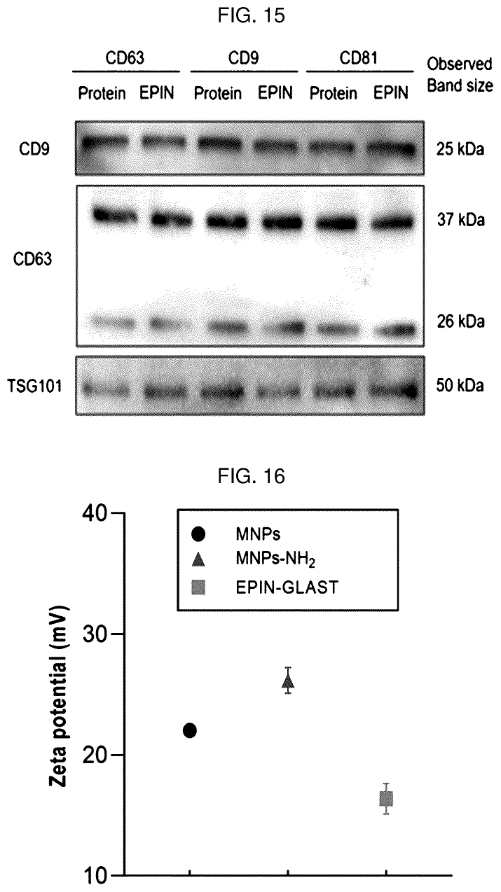

As a result, FIG. 15 shows the results of Western blotting using CD63, CD9, and CD81 antibodies for EVs isolated by EPIN and the existing protein MIP. From the results, it can be seen that in the case of EVs isolated by EPIN according to the present invention, CD63, CD9, and CD81 retained their integrity even after isolation.

### 3-3. Isolation of Astrocyte-Derived EVs by EPIN-GLAST

In order to selectively isolate astrocyte-derived EVs from biological samples of patients with a neurological disease (neuromyelitis optica spectrum disorder (NMOSD) in this example), EPIN-GLAST imprinted with the peptide selected and prepared in Example 1 was used. GLAST is EAAT1, which is a glutamate-aspartate transport protein that is present in the central nervous system.

To confirm whether EPIN-GLAST was prepared, the surface charge was measured. As a result, FIG. 16 shows that the zeta potential changed when MNPs were modified into EPIN-GLAST.

Western blotting was performed to confirm the characteristics of EVs isolated by EPIN-GLAST. EVs isolated from an HCT116 (colorectal cancer cell line) and a CCF-STTG1 (astrocyte-like cell line) cell culture medium using EPIN-GLAST were identified by EV markers CD9 and CD81 and astrocyte-specific markers EAAT1 and Aquaporin 4 (AQP4).

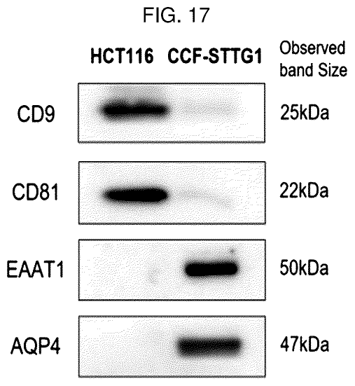

As a result, it was confirmed through FIG. 17 that, EVs were isolated from the HCT116 medium due to the positive potential of the EPIN-GLAST surface (EV surfaces generally have a negative charge), but EVs derived from specific cells (astrocytes in the case of FIG. 17) could also be isolated using the microstructure according to the present invention.

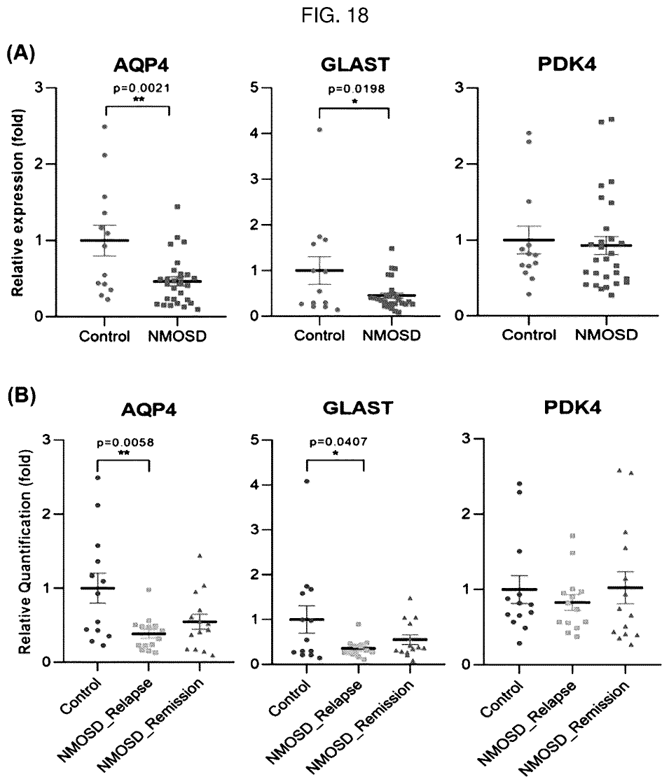

To confirm the effectiveness of EPIN-GLAST in isolating EVs from blood samples, astrocyte-derived EVs were isolated and analyzed from blood samples of healthy subjects and NMOSD patients. The results of Western blotting for AQP4, GLAST, and pyruvate dehydrogenase kinase 4 (PDK1), which are known to be associated with NMOSD, were analyzed and shown in FIG. 18A.

As a result, the expression levels of AQP4 and GLAST in the NMOSD patients were relatively lower than in the healthy subjects, indicating downregulation due to the inflammatory response of astrocytic cells.

In addition, FIG. 18B shows the results of Western blotting for AQP4, GLAST, and PDK1 after dividing the samples of NMOSD patients into the remission and relapse stages. It was confirmed that the expression levels of AQP4 and GLAST were slightly lower in the NMOSD relapse stage. This indicates severe disease activity in the relapse stage.

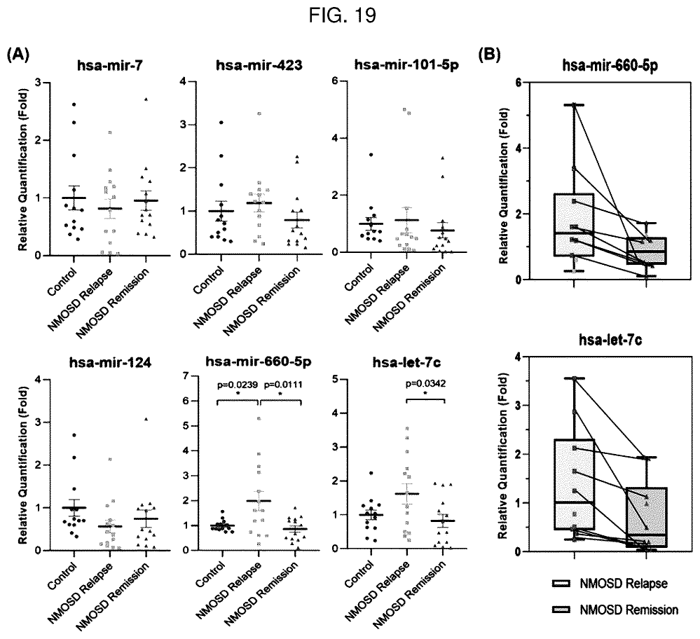

The miRNAs present in EVs perform intercellular signaling roles and may be used as biomarkers. FIG. 19A shows the results of an RT-qPCR analysis for comparing the expression levels of miRNAs in EVs isolated by EPIN-GLAST from clinical samples (healthy subjects and NMOSD patients).

As a result of confirming the expression levels of the miRNAs hsa-mir-101-5p, hsa-mir-423, hsa-mir-660-5p, hsa-mir-7, and hsa-mir-124, which are associated with NMOSD, and the miRNA has-let-7c, which is commonly used as a biomarker, it was found that the concentration of hsa-mir-660-5p increased in the relapse stage and decreased in the remission stage, which can be seen as reflecting the disease activity.

In addition, FIG. 19B compares the miRNA composition according to the disease progression stage from the same NMOSD patient, and a unique expression pattern may be confirmed according to the progression.

### 3-4. Cell-Specific EV Isolation by EPIN-MOG, EPIN-L1CAM, and EPIN-CD11b

In this example, it was confirmed whether EPIN proteins may be used to isolate EVs (exosomes) in a cell-specific manner from various neural cell lines. To this end, EPIN-MOG, EPIN-L1CAM, and EPIN-CD11b were applied to oligodendrocytes, neurons, and microglia, respectively, and the isolation of EVs was confirmed through Western blotting.

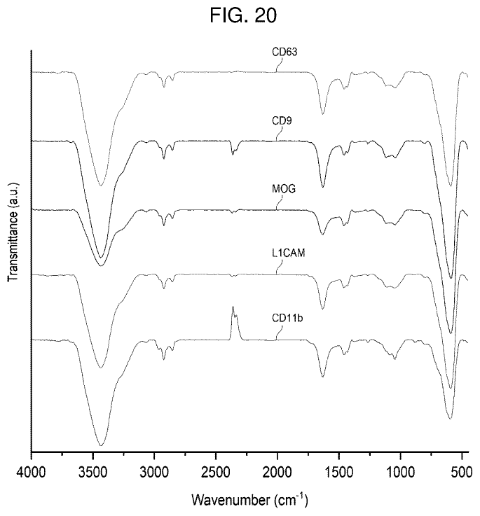

Prior to this, the changes in the FT-IR graphs of EPINs prepared with various templates were confirmed. FIG. 20 shows a diagram illustrating the FT-IR spectra for EPIN-CD63, EPIN-CD9, EPIN-L1CAM, EPIN-CD11b, and EPIN-MOG. It was confirmed that the absorption characteristics at specific wavelengths changed as EPIN interacted with each protein (CD63, CD9, MOG, L1CAM, CD11b). This suggests that each EPIN forms a unique binding pattern for each protein. These results support that EPIN is capable of selectively recognizing and binding to specific proteins, and suggest that efficient isolation and concentration of EVs can be achieved using this.

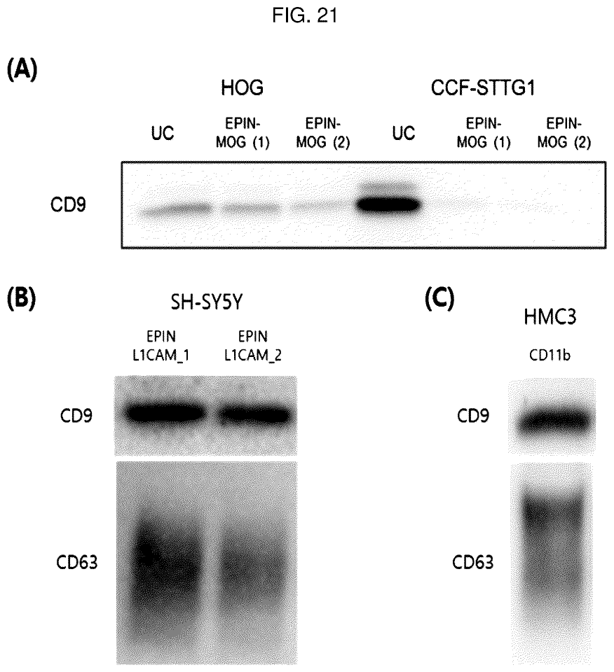

FIG. 21 shows the results of confirming the EVs isolated in a cell-specific manner using each of EPIN-MOG, EPIN-L1CAM, and EPIN-CD11b. First, EPIN-MOG was used to confirm the isolation of EVs from oligodendrocytes (HOG) and astrocytes (CCF-STTG1) (FIG. 21A). As a result of the experiment, a distinct band was confirmed for CD9, an EV marker, in the oligodendrocyte cell line (HOG) with MOG, suggesting that the EVs were successfully isolated. On the other hand, in the astrocyte cell line (CCF-STTG1) that does not express MOG, no CD9 band was observed when compared with EPIN-MOG and the comparative group of UC conditions. These results suggest that EV isolation using EPIN-MOG can be achieved in a cell-specific manner.

After isolating EVs from a neuronal cell line (SH-SY5Y) using EPIN-L1CAM, the expression of EV marker proteins was confirmed (FIG. 21B). As a result of Western blotting, clear bands were observed for the EV markers CD9 and CD63, suggesting that neuron-specific EV isolation using EPIN-L1CAM is possible.

Isolation of extracellular vesicles from microglia (MOG) was confirmed using EPIN-CD11b (FIG. 21C). As a result of Western blotting, clear bands were observed for the EV markers CD9 and CD63, confirming that microglial cell-specific EV isolation using EPIN-CD11b is possible.

The above-described series of results mean that the nanostructure according to the present invention is a technology that can be used to isolate specific cell-derived EVs from biological samples such as blood for diagnosis and analysis of diseases. Unlike conventional isolation methods, it is possible to selectively isolate EVs associated with specific diseases, so that a method of isolating and diagnosing with a small amount of sample can be provided.

The nanostructure according to the present invention includes a binding site of an EV membrane protein, so that it can selectively concentrate or isolate EVs with excellent efficiency in a short time. Since a peptide, which is small in size compared to a protein and easy to synthesize, is used as a template, the nanostructure can be prepared simply at low cost.

In addition, when an amino acid sequence of a specific cell-derived EV protein is used as a template peptide, it specifically binds to specific cell-derived EVs, so that the specific cell-derived EVs can be simply and easily isolated. In addition, since the nanostructure includes a magnetic nanoparticle, when a magnetic field is applied, a target substance can be easily and simply isolated by a magnetic force. Therefore, compared to existing technologies, it is possible to selectively concentrate or isolate EVs with excellent efficiency in a short time at low cost in a simple and easy manner.

Although the foregoing has been described with reference to preferred embodiments of the present invention, those skilled in the art will understand that the present invention can be variously modified and changed within the scope that does not depart from the spirit and scope of the present invention described in the following claims.

What is claimed is:

1. A nanostructure for isolating or concentrating extracellular vesicles, the nanostructure comprising a magnetic nanoparticle coated with a peptide-imprinted polymer.

2. The nanostructure of claim 1, wherein the magnetic nanoparticle and the peptide-imprinted polymer are linked by an amide bond.

3. The nanostructure of claim 1, wherein the magnetic nanoparticle is an iron oxide (Fe₃O₄) nanoparticle.

4. The nanostructure of claim 1, wherein the nanostructure has a diameter of 100 to 200 nm.

5. The nanostructure of claim 1, wherein the peptide consists of 8 to 20 amino acids.

6. The nanostructure of claim 1, wherein the peptide is derived from an extracellular vesicle membrane protein of a brain cell.

7. The nanostructure of claim 6, wherein the brain cell is one or more selected from an astrocyte, a neuron, a microglial cell, and an oligodendrocyte.

8. The nanostructure of claim 6, wherein the peptide consists of any one of SEQ ID NOs: 4 to 7.

9. The nanostructure of claim 1, wherein the polymer is polymerized from one or more monomers selected from styrene, N-(3-aminopropyl) methacrylamide, N-isopropylacrylamide, methacrylic acid, ethylene glycol dimethacrylate, N-tert-butylacrylamide, N,N-dimethylaminopropyl acrylamide, and acrylamide.

10. The nanostructure of claim 9, wherein, during the polymerization, one or more selected from acrylic acid, N,N-methylenebis(acrylamide), ammonium persulfate, N,N,N′,N′-tetramethylethylenediamine, and benzoyl peroxide are further added.

11. The nanostructure of claim 1, wherein the nanostructure is for isolating or concentrating extracellular vesicles in a blood or cell culture medium sample.

12. The nanostructure of claim 1, wherein the nanostructure is for isolating or concentrating extracellular vesicles derived from a brain cell.

13. A method of isolating or concentrating extracellular vesicles using the nanostructure of claim 1.

14. The method of claim 13, comprising:
(a) a step of bringing the nanostructure into contact with extracellular vesicles; and
(b) a step of applying a magnetic field.

15. The method of claim 14, further comprising:
(c) a step of isolating a material captured by the magnetic force from the magnetic field; and
(d) a step of isolating the extracellular vesicles from the nanostructure.

16. A method of preparing the nanostructure of claim 1, the method comprising:
(i) a step of treating a magnetic nanoparticle with ammonia water;
(ii) a step of treating the product of Step (i) with a peptide;
(iii) a step of treating the product of Step (ii) with a monomer compound;
(iv) a step of polymerizing the monomer compound of the product of Step (iii) into a polymer; and
(v) a step of removing the peptide from the product of Step (iv).
